(19) United States
(12) Patent Application Publication
(10) Pub. No.: US 2026/0240534 A1
(43) Pub. Date: Aug. 20, 2026

(54) Knotless Locking Constructs and Methods of Tissue Repair

(71) Applicant: Arthrex, Inc., Naples, FL (US)

(72) Inventors: Justin M. BOYLE, Naples, FL (US); Jacob A. JOLLY, Naples, FL (US); Bertrand SONNERY-COTTET, Naples, FL (US)

(73) Assignee: Arthrex, Inc., Naples, FL (US)

(21) Appl. No.: 19/468,056

(22) Filed: Feb. 3, 2026

Related U.S. Application Data
(60) Provisional application No. 63/752,992, filed on Feb. 3, 2025.

(51) Int. Cl. A61B 17/04

(52) U.S. Cl. CPC A61B 17/0401; A61B 2017/0445; A61B 2017/0454

(57) ABSTRACT

Surgical anchor constructs, fixation devices, and methods for tissue repairs are disclosed. A soft suture anchor has a cannulated sheath (that is fixed in bone) and a plurality of repair sutures attached to the sheath and forming a plurality of knotless loops. Soft tissue is secured to bone with two or more knotless loops that secure the soft tissue similar to a cam buckle strap.

### CROSS-REFERENCE TO RELATED APPLICATIONS

The application claims priority to US Provisional Patent Application 63/752,992 filed Feb. 3, 2025, the disclosure of which is hereby incorporated by reference in its entirety herein.

### BACKGROUND

The disclosure relates to the field of surgery and, more specifically, to knotless anchor constructs and associated methods of tissue repairs.

### SUMMARY

Reconstruction systems, assemblies and methods for fixation of tissue are disclosed.

A surgical construct may create a knotless, self-locking, reinforced repair. A surgical construct may be made completely of suture to achieve fixation in bone without a separate anchoring body. A surgical construct may be employed in knotless fixation of first tissue to second tissue, for example, fixation of soft tissue to bone. A surgical construct may be a knotless, tensionable construct that includes one or more soft anchors in the form of all-suture anchors. In some implementations, the all-suture anchors may be single-loaded or multiple-loaded.

In some implementations, the soft anchor may be a loaded construct that includes a soft anchor sheath with two or more knotless mechanisms having two or more repair sutures configured to fold soft tissue back on itself similar to a cam buckle strap. The two or more knotless loops compress and fixate soft tissue to another tissue in a manner similar to that of a cam buckle strap. Folding of the soft tissue creates additional friction and a pinching of the tissue that prevents the soft tissue from sliding back through the loops.

Methods of tissue repairs are also disclosed. In some implementations, knotless surgical constructs provide tissue fixation without any knot formation, by providing an all-suture repair which is conducted with fewer steps, increased anchor fixation and suture management, and increased reproducibility.

These and other features and advantages of this disclosure will become apparent and will be understood from the following detailed description of the various aspects of the disclosure taken in conjunction with the accompanying drawings.

### BRIEF DESCRIPTION OF THE DRAWINGS

FIG. 1 illustrates a surgical repair site.

FIG. 2 illustrates a surgical construct.

FIGS. 3-8 illustrate schematic steps of an exemplary surgical repair with the surgical construct of FIG. 2 at the repair site of FIG. 1.

FIGS. 9-15 illustrate schematic steps of another exemplary surgical repair with the surgical construct of FIG. 2.

FIGS. 16 and 17 illustrate additional views of surgical repair of FIG. 15.

### DETAILED DESCRIPTION

Tensionable, knotless, self-locking surgical constructs can create knotless, reinforced repairs.

A soft tissue repair system may include one or more knotless, tensionable constructs. In some implementations, a knotless, tensionable construct may include an all-suture anchor that is multiple loaded to provide multiple knotless fixation points and locations.

In some implementations, a knotless, tensionable construct may include a soft suture anchor with (i) an anchor sheath; and (ii) two or more knotless mechanisms including two or more knotless loops formed by repair sutures (flexible strands) attached to the anchor sheath. The construct may include at least one shuttle/pull device attached to the anchor sheath. Once the knotless mechanisms have been converted creating their corresponding knotless loops, soft tissue may be pulled through a first loop and cinched down. A second knotless repair mechanism may be used to cinch down the remaining excess soft tissue after it has been folded back to itself, thus eliminating the need for having to stitch the soft tissue back into itself.

Methods of knotless tissue repairs are also disclosed. In some implementations, a surgical construct provides knotless tissue to tissue fixation, for example, knotless fixation of soft tissue (ligament, tendon, graft, etc.) to bone without any knot formation and in a simple and fast manner. The tensionable repair with all-suture anchors facilitate knotless onlay fixation of soft tissue (tendon, ligament, graft, etc.) to bone. Soft tissue may be pulled through an adjustable loop of a knotless repair mechanism of a soft suture anchor and cinched down. The soft tissue may then be folded back on itself and fixated with an adjustable loop of another knotless mechanism. In this manner, the need to stitch the soft tissue back into itself is eliminated, as well as any tissue irritation typically associated with hard metal staples.

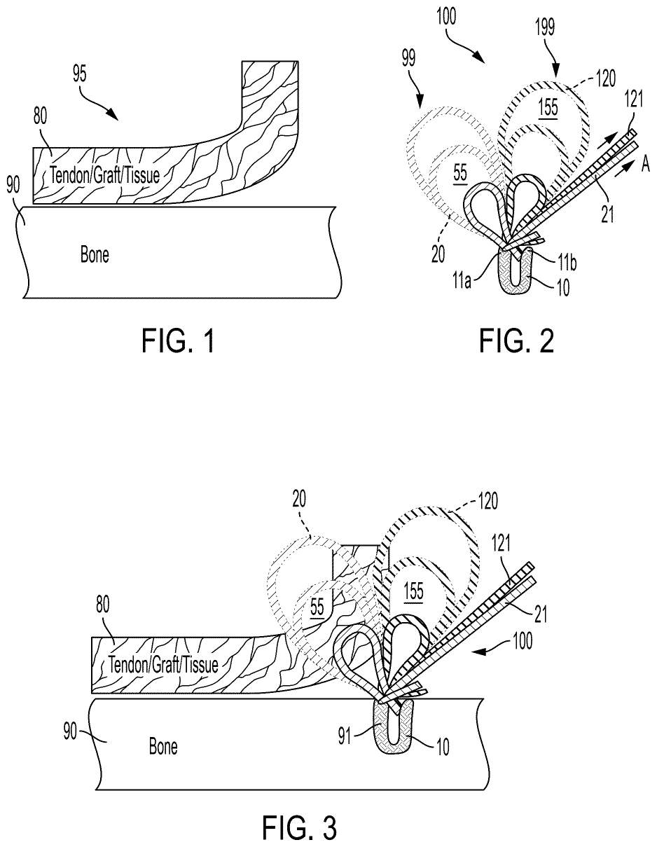

Referring now to the drawings, where like elements are designated by like reference numerals, FIGS. 1 and 2 illustrate an exemplary repair site 95 undergoing soft tissue repair with exemplary fixation devices 100. FIGS. 3-17 illustrate steps of exemplary soft tissue repairs 101, 201, 301 with exemplary fixation device 100.

Fixation device 100 may be a soft anchor. Fixation device 100 may be a soft suture anchor construct. Fixation device 100 may be a surgical construct. Fixation device 100 may be a tensionable, knotless, self-locking, surgical construct. As detailed below, fixation device 100 is an implantable device made completely of suture or sutures that achieve fixation in bone without a separate anchoring body. The design enables fixation by deployment into bone with one or more suture tails remaining outside the bone for device attachment, pulling, bunching, tensioning and/or alternative usage.

FIG. 1 illustrates a schematic view of surgical repair site 95 with a first tissue 80 to be attached to a second tissue 90. Tissue 80 may be soft tissue such as tendon, graft, ligament, etc. Tissue 90 may be bone.

FIG. 2 illustrates surgical construct 100 which includes (i) a soft anchor 50 with cannulated sheath 10; and (ii) at least a first flexible strand 20 and at least a second flexible strand 120. The at least one flexible strand 20, 120 may be a repair suture. Construct 100 may also include a shuttle/pull device (not shown) to aid in passing of each of the flexible strand 20, 120 through the cannulated sheath 10 and through itself, to allow formation of a splice and corresponding adjustable, knotless, closed, continuous loop.

Cannulated sheath 10 of soft anchor 50 can be in the form of a tubular sleeve or tubular member with a body made of a flexible material, such as a braided, woven, or knitted structure made of yarns, fibers, filaments, sutures or similar materials, or combinations of these materials. In some implementations, sheath 10 may be constructed of ultra-high molecular weight polyethylene (UHMWPE). In some implementations, sheath 10 may be constructed of UHMWPE and polyester. In some implementations, sheath 10 may be constructed of UHMWPE with an elastic component. In some implementations, sheath 10 may be constructed of polyester suture material with an elastic component. The elastic component may be elastane. The elastic component may be incorporated into the sheath via braiding, weaving, and/or knitting. In some implementations, an elastic sheath may include a combination of elastic, polyester, and UHMWPE all braided in a tubular jacket. An elastic sheath may consist essentially of elastane, polyester, and UHMWPE. The elastic component provides elasticity while the other components provide strength and limit the elongation of the suture.

Sheath 10 can also be any tubular or non-tubular structure having a lumen along its length. The lumen can have a constant diameter. The lumen can have a varying diameter along its length.

Tubular body of sheath 10 extends between opposing ends 11a, 11b. The opposing ends 11a, 11b can be open or closed ends. The tubular body establishes the bore or lumen that extends between the opposing ends 11a, 11b. Flexible strands 20, 120 (flexible coupler 20, 120; suture 20, 120; repair suture 20, 120) may be passed through at least a portion of bore of the sheath 10 and through the body of the sheath 10 with an exemplary free end 21, 121 exiting one of the ends 11a, 11b, or exiting at a location close to the one of the free ends 11a, 11b. Flexible strand 20, 120 located within the body of the sheath 10 may be fixed relative to the sheath (or affixed to the sheath 10), for example, by being stitched to the sheath 10, or may slide through the sheath 10.

FIG. 2 illustrates an exemplary-only implementation of soft anchor 50 with flexible strands 20, 120 passing through the sheath 10 and with only one free end 21, 121 exiting the sheath 10. Flexible strands 20, 120 are part of knotless repair mechanisms 99, 199 which include splices formed by passing of the flexible strands 20, 120 back through the sheath 10 and through themselves to form at least one splice and loop 55, 155. Tensioning of the free end 21, 121 may be conducted by pulling end 21, 121 in the direction of arrow A to allow loop 55, 155 to decrease its length and diameter and tension the final repair. Loop 55, 155 may be an adjustable, flexible, tensionable, closed, knotless loop formed around or through soft tissue 80 to be secured to the bone 90, and as detailed below.

Soft anchor 50 may also include a shuttle/pull device (not shown in FIG. 2) to aid in the formation of at least one loop 55, 155 and corresponding splice. The shuttle/pull device may be a suture passing instrument, a suture passer, a shuttle link, a FiberLink™, a nitinol loop. Shuttle/pull device may include an eyelet/loop for passing flexible coupler 20, 120. If more than one shuttle/pull devices are provided, the shuttle/pull devices can extend through the sheath 10 in similar or different directions and/or orientations and/or locations. The at least one loop 55, 155 mat be an adjustable, knotless, closed, self-cinching, continuous loop with an adjustable perimeter and/or length.

In some implementations, soft suture anchor 50 may be a knotless FiberTak® soft anchor that may be a double-loaded or multiple-loaded anchor. In some implementations, soft suture anchor 50 may be a knotless FiberTak® soft anchor that may be a hybrid FiberTak® soft anchor with a shuttle/pull device that acts both as a shuttling suture and as a repair suture. In some implementations, soft suture anchor 50 may be a knotless FiberTak® soft anchor with repair sutures that may be furcated, bifurcated, trifurcated, etc. In some implementations, soft suture anchor 50 may be a knotless FiberTak® soft anchor with a plurality of repair sutures 20, 120 and a plurality of corresponding shuttle/pull devices. The number of the repair sutures 20, 120 and the number of the shuttle/pull devices may be similar or different. Details of an exemplary soft suture anchor with a soft anchor sleeve (sheath or tubular member) and flexible shuttling strands are set forth, for example, in U.S. application Ser. No. 15/998,516 entitled “Methods of Tissue Repairs” filed on Aug. 16, 2018 (issued as U.S. Pat. No. 10,849,734 on Dec. 1, 2020), the disclosure of which is incorporated by reference in its entirety herein. The flexible couplers and the one or more shuttle/pull devices can extend through the sleeve in similar or different directions and/or orientations and/or locations.

Although the embodiments and implementations detailed above have been described with reference to a soft anchor 50 provided with two repair sutures 20, 120, it must be understood that the disclosure is not limited to this exemplary-only implementation and contemplates soft anchors provided with any number of repair sutures, depending on the specifics and characteristics of the surgical repair and the surgeon's preferences.

Reference is now made to FIGS. 3-8 which illustrate schematic steps of exemplary soft tissue repair 101 that allows tissue 80 to be attached to tissue 90 with exemplary surgical construct 100 of FIG. 2. The all-suture anchor repair 101 is a tensionable, knotless repair that facilitates smaller number of drill holes, fewer surgical steps, elimination of tissue irritation due to the use of hard metal devices, and increased fixation of soft tissue to bone. Repair 101 is conducted with a double loaded knotless locking construct (a FiberStaple double knotless locking construct) such as construct 100 detailed above. As detailed below, the novel method enables the surgeon to fixate tissue against bone using two or more knotless mechanisms in a single anchor to fold a graft back on itself in a similar manner to a cam buckle strap. The folding of the graft creates additional friction and pinching of the tissue that prevents the tissue from sliding back through the loop. The novel method allows fixation of a graft that folds back over onto itself with two or more knotless loops that compress and fixate the tissue to bone in a similar manner to a cam buckle strap.

The implementation below will be detailed with reference to a specific ligament repair of the knee. However, soft anchor 100 is configured for use in various soft tissue repairs or fixations and can be fixated inside bone or partially inside bone for attaching tissue (e.g., ligament, tendon, graft, etc.) to bone. For example, soft anchor 100 may be used in conjunction with a wide variety of orthopedic surgical repairs, including but not limited to rotator cuff repairs, Achilles tendon repairs, patellar tendon repairs, ACL/PCL reconstructions, hip, wrist, and shoulder reconstructions, among many others. The fixation can be on or over bone.

FIG. 3: Drill a pilot hole 91 in bone 90; pilot hole 91 may be an exemplary 2.6 mm pilot hole. Insert construct 100 into pilot hole 91.

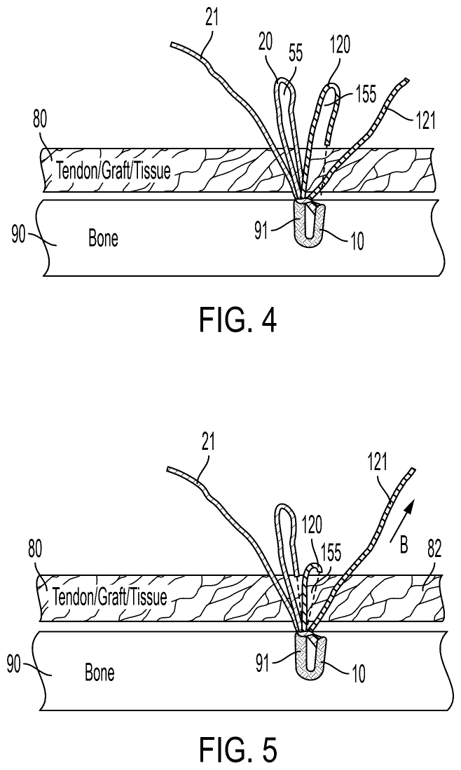

FIG. 4: Pull graft 80 through both loops 55, 155 of pre-converted repair sutures 20, 120.

FIG. 5: The knotless repair suture 120 towards end 82 of soft tissue 80 is tensioned first, approximating the soft tissue 80 in the anatomical position. End 121 of repair suture 120 is pulled in the direction of arrow B to decrease the perimeter and length of loop 155.

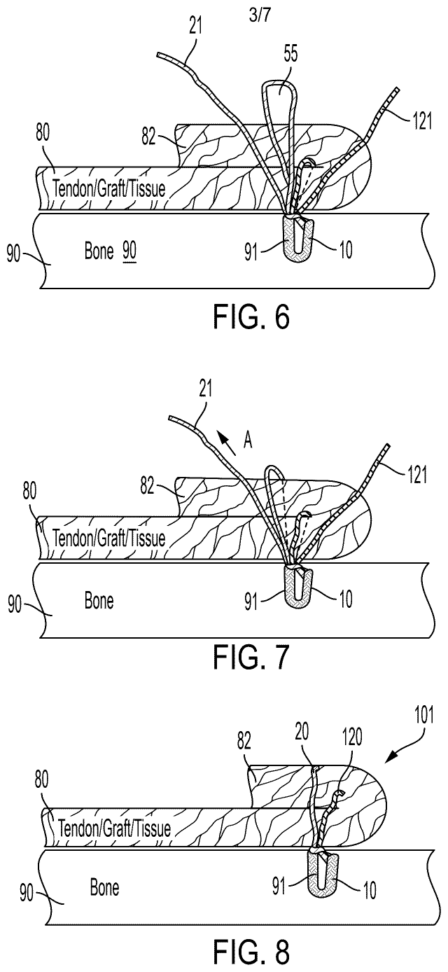

FIG. 6: After tensioning of the repair suture 120, graft 80 is folded back onto itself and passed back through the second knotless repair loop 55, forming a folded graft tissue. By folding graft 80 over onto itself, the thickness of the folded graft through loop 55 is about double from the thickness of unfolded graft 80.

FIG. 7: The second knotless repair loop 55 is fully tensioned to create 180 run of the graft on itself to lock and improve fixation.

FIG. 8: Exemplary repair 101 with construct 100 of the present disclosure employed for soft tissue to bone reattachment or approximation is shown in FIG. 8. Graft 80 is attached to bone in a double bundle reconstruction with the aid of repair sutures 20, 120 of surgical construct 100 secured within a single hole 91 in bone 90. A first portion of graft 80 is secured with a first knotless loop 155 and a second portion of graft 80 (folded graft portion) is subsequently secured with a second knotless loop 55.

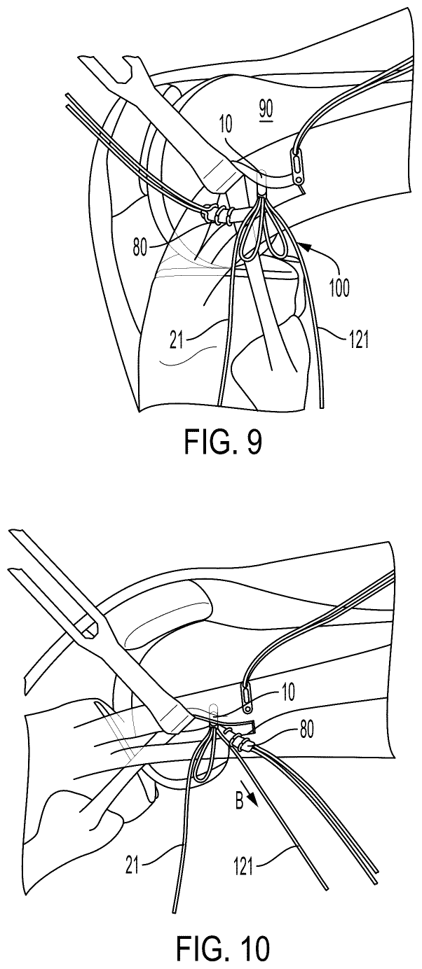

Reference is now made to FIGS. 9-15 which illustrate subsequent steps of exemplary repair 201 with surgical construct 100 detailed above. Repair 201 is a knotless repair of the knee conducted with graft 80 to be attached to femur 90 with double loop construct 100.

FIG. 9: Surgical construct 100 is illustrated adjacent femoral graft 80 to be secured to femur 90.

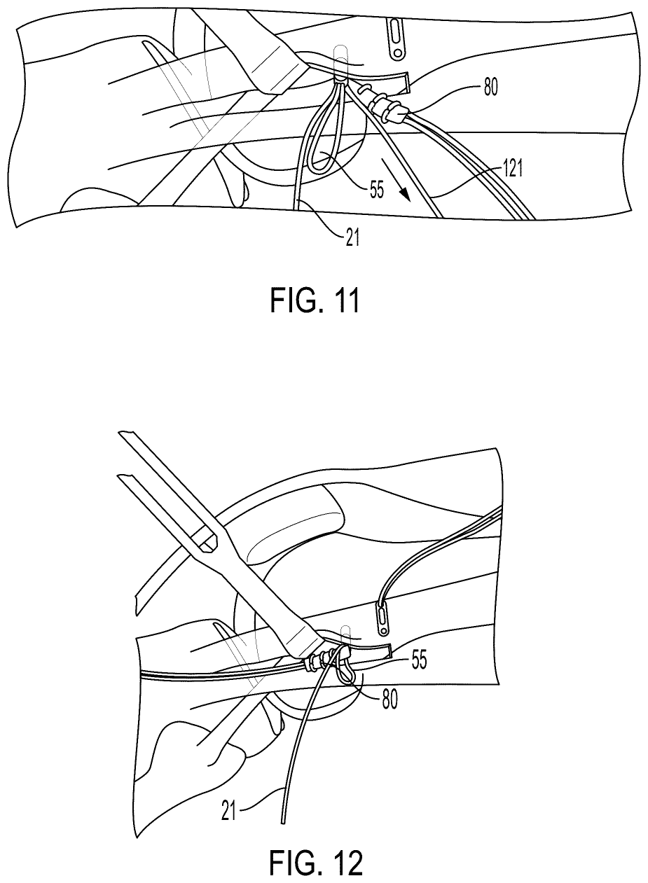

FIGS. 10 and 11: Graft 80 is passed through both loops 55, 155 of construct 100. End 121 of repair suture 120 is pulled in the direction of arrow B to decrease the perimeter and length of knotless loop 155 and to approximate the graft in the anatomical position.

FIG. 12: After tensioning of the repair suture 120, graft 80 is folded back onto itself and passed back through the second knotless repair loop 55.

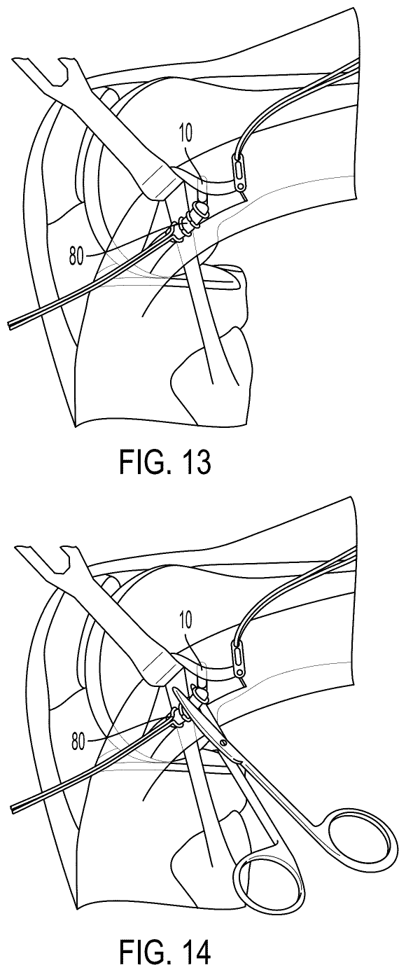

FIGS. 13 and 14: The second knotless repair loop 55 is tensioned to lock the graft and improve overall fixation.

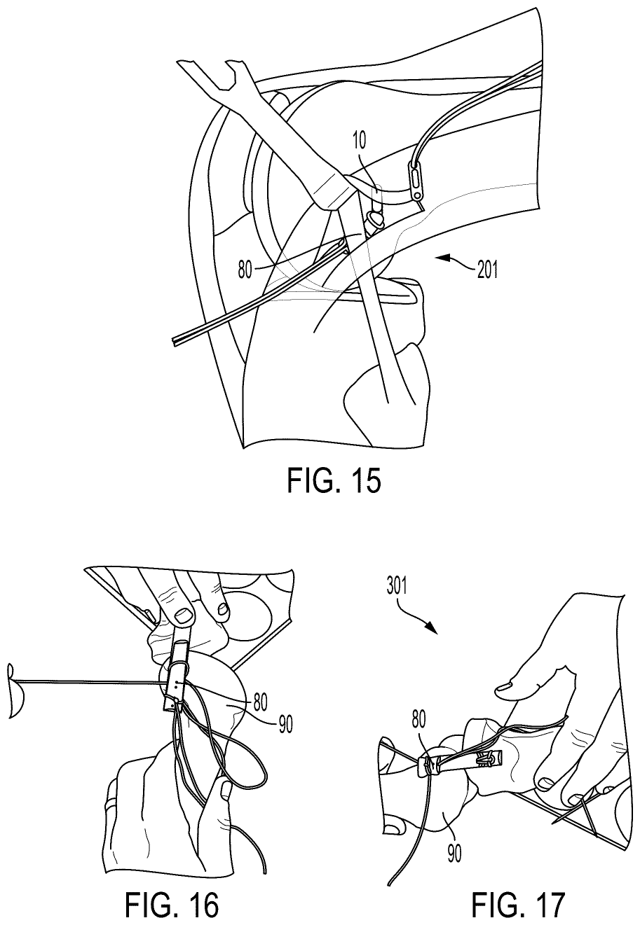

FIG. 15: Exemplary repair 201 with construct 100 of the present disclosure employed for knee repair is shown in FIG. 15. Graft 80 is attached to bone in a double bundle reconstruction with the aid of repair sutures 20, 120 of surgical construct 100 secured within a single hole in bone 90.

FIGS. 16 and 17 illustrate additional views of knee repair 301 wherein graft 80 is folded once over itself and secured with two or more knotless loops 55, 155 of surgical construct 100 on femur 90.

Although the implementations above have been detailed with reference to soft tissue 80 attached to bone 90, it must be understood that the disclosure is not limited to these exemplary-only embodiments. Accordingly, the disclosure also contemplates embodiments wherein surgical construct 100 may provide bone-to-bone fixation such as repairs associated with patellar fractures, for example.

Further, although the implementations above have been detailed with reference to a single length of soft tissue 80 secured by two knotless loops of two separate knotless mechanisms of a single suture anchor, it must be understood that the disclosure is not limited to these exemplary-only embodiments. Thus, the disclosure also contemplates embodiments wherein more than two knotless loops are employed. The multiple knotless loops may be part of a single anchor sheath or may correspond to multiple anchor sheaths. Accordingly, the disclosure also contemplates embodiments and implementations wherein a plurality of soft anchors with multiple knotless mechanisms and multiple strands and knotless loops are employed for fixation of a first tissue to a second tissue by the methods described above. In addition, single-loaded knotless anchors may be also employed to fixate tissue 80 to tissue 90. A plurality of single-loaded anchors provides single knotless loops that are employed for graft folding and fixation similar to the cam buckle strap mechanism detailed above. If single loaded anchors are employed, the flexible ends may be provided with attached needles or similar structures.

The disclosure has also applications to repairs wherein two or more portions or lengths of soft tissue are attached to another tissue such as bone. In these implementations, the two or more lengths of soft tissue can overlap and then can be passed through the multiple loops of construct 100 to undergo the steps detailed above.

If more than two flexible couplers 20, 120 are employed, the plurality of flexible couplers can reside within and around the cannulated sheath 10 independent from each other. The flexible couplers may be similar to each other or different from each other. In some implementations, any of flexible coupler 20, 120 can be bifurcated or furcated from the sheath 10 to provide a plurality of loops.

In some implementations, the flexible coupler 20, 120 may come out of the cannulated sheath 10 and be braided to a smaller diameter round suture which may be passed back through the sheath. The braided suture may be furcated/branched/forked (for example, bifurcated or trifurcated, etc.) to provide a plurality of passing sutures. In some implementations, the end of flexible coupler 20, 120 may be a tapered end. In some implementations, flexible coupler 20, 120 may be a single strand suture. In some implementations, flexible coupler 20, 120 may be a single braided suture.

The constructs, systems, and assemblies of the present disclosure may be employed in numerous knotless soft tissue repairs and fixations, for example, fixation of soft tissue to bone. In some implementations, a surgical construct 100 may provide first tissue to second tissue fixation, for example, fixation of soft tissue 80 (ligament, tendon, graft, etc.) to bone 90 in a simple and fast manner.

The exemplary fixation device 100 detailed above are soft anchors formed of “soft” materials, such as suture materials, yarns, fibers, filaments, strings, fibrils, strands, etc., or any combination of such materials. The soft materials may be synthetic or natural materials, or combinations of synthetic and natural materials, and may be bio-degradable or non-degradable, and may be elastic or non-elastic within the scope of this disclosure. These soft materials confer the ability to be inserted into bone sockets/holes/tunnels and bunch together, collapse, expand and/or change shape to fixate within the socket/hole/tunnel. In some implementations the soft anchor 100 is made exclusively of soft, suture-based materials. Soft anchor 100 may be an “all-suture” construct.

Soft anchor 100 is configured for use in various soft tissue repairs or fixations and may be fixated inside bone or over bone for attaching tissue (e.g., ligament, tendon, graft, etc.) to bone. For example, the soft anchor 100 can be used in conjunction with a variety of orthopedic surgical repairs, including but not limited to rotator cuff repairs, Achilles tendon repairs, patellar tendon repairs, ACL/PCL reconstructions, wrist, hip and shoulder reconstructions, among many others. The fixation can be on or over bone.

A soft suture anchor 100 comprises: (i) a cannulated sheath 10 having a length, a first end 11a, and a second end 11b; and (ii) at least two flexible couplers 20, 120 attached to the cannulated sheath 10 and forming two knotless, tensionable loops 55, 155 around tissue 80 to be attached to bone 90. At least one of the flexible couplers 20, 120 may be formed of elastic suture.

Methods of soft tissue repair which do not require tying of knots and allow adjustment of both the tension of the suture and the location of the tissue with respect to the bone, while providing a self-locking mechanism, are disclosed.

A method of knotless tissue repair 101, 201, 301 comprises inter alia the steps of: (i) inserting a tubular sheath 10 of a suture anchor 50 of a surgical construct 100 into hole 91 in a first tissue 90; (ii) passing a second tissue 80 through loops 55, 155 of surgical construct 100; (iii) tensioning one of the loops 55, 155 to approximate the second tissue 80 to the first tissue 90; (iv) folding the second tissue 80 over itself and over the tensioned loop and back through the other of the loops 55, 155 to form a folded tissue; and (v) tensioning the other of the loops 55, 155 to secure the folded tissue to the first tissue 90. The method may further comprise forming at least two adjustable, knotless, tensionable loops 55, 155 with flexible strands 20, 120 and with one or more shuttle/pull devices. The method may further comprise employing the flexible coupler 20, 120 for additional surgical procedures. The additional surgical procedures may include passing the flexible coupler 20, 120 through or around tissue 80 multiple times; and/or employing the flexible coupler 20, 120 with additional fixation devices. The additional fixation devices can be any of a knotless anchor, a hard body anchor, or a soft anchor.

The knotless tissue repair and reconstruction can be any of small joint repairs, rotator cuff repairs, Achilles tendon repair, patellar tendon repair, ACL/PCL reconstruction, wrist, elbow, hip and shoulder reconstruction procedures, and applications for elastic suture used in or with suture anchors. The surgical constructs and repair methods of the present disclosure can be employed in any tissue repairs that may not involve knot tying.

Any of sheath 10 and repair suture 20, 120 can include a flexible material, for example, multifilament, braided, knitted, woven suture, or can include fibers of ultrahigh molecular weight polyethylene (UHMWPE) or the FiberWire® suture (disclosed in U.S. Pat. No. 6,716,234, the disclosure of which is hereby incorporated by reference in its entirety herein).

Repair suture 20, 120 may be any flexible coupler or flexible strand and may be formed of various flexible materials and strands such as round suture, flat suture, ribbon, or flat tape (for example, suture tape) or combination of suture and tape. Exemplary materials may include suture, silk, cotton, nylon, polypropylene, polyethylene, ultrahigh molecular weight polyethylene (UHMWPE), polyethylene terephthalate (PET), and polyesters and copolymers thereof, or combinations thereof. Repair suture 20, 120 may have cross-sections of various forms and geometries, including round, oval, rectangular, or flat, among others, or combination of such forms and geometries.

In some implementations, repair suture 20, 120 may be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N. V., Heerlen, the Netherlands), braided with at least one other fiber, natural or synthetic, to form lengths of suture material. Repair suture 20, 120 may be braided or multi-filament suture such as FiberTape® suture tape (as disclosed in U.S. Pat. No. 7,892,256, the disclosure of which is incorporated in its entirety herewith). Repair suture 20, 120 may include elastic material. Repair suture 20, 120 may consist essentially of elastic suture.

Repair suture 20, 120 can be also formed of a stiff material, or combination of stiff and flexible materials, particularly for the regions of the coupler that may need to be passed/spliced through the body of the coupler and depending on whether they are employed with additional fixation devices.

Various structural elements of surgical construct 100 including sheath 10 and repair suture 20, 120 may be visually coded, making identification and handling of the sheath and suture legs simpler. Easy identification of suture in situ is advantageous in surgical procedures, particularly during arthroscopic surgeries, endoscopic and laparoscopic procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed:

1. A tissue-to-bone repair comprising:
a soft anchor secured to bone, the soft anchor comprising a cannulated sheath having a length, a first end, a second end, and at least two repair sutures attached to the cannulated sheath and forming at least two knotless loops;
a first tissue portion secured to the bone by one of the at least two knotless loops; and
a second tissue portion secured to the bone by another of the at least two knotless loops.

2. The tissue-to-bone repair of claim 1, wherein the first tissue portion has a first height and wherein the second tissue portion has a second height, and wherein the first height is different from the second height.

3. The tissue-to-bone repair of claim 2, wherein the second height is about twice greater than the first height.

4. The tissue-to-bone repair of claim 1, wherein each of the at least two repair sutures has a first end and a second end, and wherein each of the at least two repair sutures is configured to extend through the at least a portion of the cannulated sheath and along its length, and wherein the first end and the second end exit the cannulated sheath at a location along the length of the cannulated sheath.

5. The tissue-to-bone repair of claim 1, wherein the cannulated sheath is a tubular sheath secured into the bone, wherein a free end of each of the at least two repair sutures passes through the tubular sheath at least once to form a splice and one of the at least two loops.

6. The tissue-to-bone repair of claim 5, wherein the one of the at least two loops is an adjustable, flexible, tensionable, knotless loop with an adjustable perimeter and length.

7. The tissue-to-bone repair of claim 1, wherein the soft anchor is an all-suture soft anchor and the at least two repair sutures are round suture.

8. The tissue-to-bone repair of claim 1, wherein the cannulated sheath is elastic.

9. A method of tissue repair, comprising:
inserting a cannulated sheath of a soft suture anchor into a first tissue; and
securing a second tissue to the first tissue by employing a plurality of knotless loops of the soft suture anchor.

10. The method of claim 9, further comprising:
securing the second tissue to the first tissue with one of the plurality of knotless loops;
subsequently, folding the second tissue over itself to form a folded tissue; and
securing the folded tissue to the first tissue with another of the plurality of knotless loops.

11. The method of claim 10, wherein the soft suture anchor includes
a plurality of flexible couplers attached to the cannulated sheath, each of the plurality of flexible couplers being configured to pass back through the cannulated sheath and through itself to form a plurality of splices and the plurality of knotless loops, and wherein the method further comprises:
passing a first flexible coupler through the cannulated sheath to form a first splice and a first knotless loop;
passing a second flexible coupler through the cannulated sheath to form a second splice and a second knotless loop; and
passing the second tissue through the first knotless loop and through the second knotless loop.

12. The method of claim 11, further comprising:
pulling one end of the first flexible coupler to secure the second tissue to the first knotless loop;
folding the second tissue over itself to form the folded tissue; and
pulling one end of the second flexible coupler to secure the folded tissue to the second knotless loop.

13. The method of claim 12, wherein pulling the first flexible coupler and pulling the second flexible coupler are conducted in about same direction.

14. The method of claim 12, wherein each of the first knotless loop and the second knotless loop is a closed, continuous, tensionable, adjustable loop formed around, through, or over the second tissue.

15. The method of claim 9, wherein the first tissue is bone, and the second tissue is soft tissue.

16. The method of claim 9, wherein the first tissue is bone and the second tissue is tendon, ligament or graft.

17. The method of claim 9, wherein the tissue repair is a knee repair.

18. A method of knotless tissue repair, comprising:
inserting a cannulated sheath of a soft suture anchor into a first tissue, the soft suture anchor including a first knotless loop and a second knotless loop, wherein the first knotless loop is independently formed from the second knotless loop;
passing a second tissue through the first knotless loop and through the second knotless loop;
cinching the first knotless loop to approximate the second tissue to the first tissue;
subsequently, folding the second tissue over itself at least once to form a folded tissue;
passing the folded tissue through the second loop; and
cinching the second knotless loop to approximate the folded tissue to the first tissue.

19. The method of claim 18, wherein the knotless tissue repair is one of knee, wrist, hand, elbow or shoulder repair.

20. A method of knotless tissue repair, comprising:
inserting a cannulated sheath of a first soft suture anchor into a first tissue, the first soft suture anchor including a first knotless loop;
inserting a cannulated sheath of a second soft suture anchor into the first tissue, the second soft suture anchor including a second knotless loop;
passing a second tissue through the first knotless loop and through the second knotless loop;
cinching the first knotless loop to approximate the second tissue to the first tissue;
subsequently, folding the second tissue over itself at least once to form a folded tissue;
passing again the folded tissue through the second knotless loop; and
subsequently, cinching the second knotless loop to approximate the folded tissue to the first tissue.
